(19) United States
(12) Patent Application Publication
(10) Pub. No.: US 2025/0387611 A1
(43) Pub. Date: Dec. 25, 2025

(54) STROKE VOLUME IMPROVEMENT DEVICES

(71) Applicant: EDWARDS LIFESCIENCES CORPORATION, Irvine, CA (US)

(72) Inventor: David Maimon, Atlit (IL)

(21) Appl. No.: 19/271,003

(22) Filed: Jul. 16, 2025

Related U.S. Application Data
(63) Continuation of application No. PCT/US2024/013393, filed on Jan. 29, 2024.
(60) Provisional application No. 63/482,281, filed on Jan. 30, 2023.

(51) Int. Cl. A61M 60/139; A61M 60/295; A61M 60/468; A61M 60/841

(52) U.S. Cl. CPC A61M 60/139; A61M 60/295; A61M 60/468; A61M 60/841; A61M 2205/04; A61M 2210/125; A61M 2210/127

(57) ABSTRACT

A cardiac implant comprises a first fluid transfer device configured for placement at least partially within a blood vessel of a heart, a second fluid transfer device configured for placement outside the blood vessel, and one or more lines interconnecting the first fluid transfer device and the second fluid transfer device.

### RELATED APPLICATION

This application is a continuation of International Patent Application No. PCT/US2024/013393, filed Jan. 29, 2024, which claims the benefit of U.S. Provisional Application No. 63/482,281, filed Jan. 30, 2023, the complete disclosures of which are hereby incorporated by reference in their entireties.

### BACKGROUND

The present disclosure generally relates to the field of medical implant devices and procedures. Insufficient or reduced compliance in certain blood vessels, including arteries such as the aorta, can result in reduced perfusion, cardiac output, and other health complications. Increasing compliance and/or otherwise controlling flow in such blood vessels can improve patient outcomes.

### SUMMARY

Some implementations of the present disclosure relate to a cardiac implant including: a first fluid transfer device configured for placement at least partially within a blood vessel of a heart; a second fluid transfer device configured for placement outside the blood vessel; and one or more lines interconnecting the first fluid transfer device and the second fluid transfer device.

In some aspects, the techniques described herein relate to a cardiac implant, wherein the first fluid transfer device includes a first inflatable balloon.

In some aspects, the techniques described herein relate to a cardiac implant, wherein the one or more lines include a fluid tube.

In some aspects, the techniques described herein relate to a cardiac implant, wherein the second fluid transfer device includes a second inflatable balloon.

In some aspects, the techniques described herein relate to a cardiac implant, wherein the fluid tube is configured to convey a fluid between the first inflatable balloon and the second inflatable balloon.

In some aspects, the techniques described herein relate to a cardiac implant, wherein deflation of the first inflatable balloon causes inflation of the second inflatable balloon.

In some aspects, the techniques described herein relate to a cardiac implant, wherein deflation of the second inflatable balloon causes inflation of the first inflatable balloon.

In some aspects, the techniques described herein relate to a cardiac implant, wherein the first fluid transfer device includes a first pump.

In some aspects, the techniques described herein relate to a cardiac implant, wherein the one or more lines include one or more electrical wires.

In some aspects, the techniques described herein relate to a cardiac implant, wherein the second fluid transfer device includes a second pump.

In some aspects, the techniques described herein relate to a cardiac implant, wherein the one or more electrical wires are configured to convey electrical signals between the first pump and the second pump.

In some aspects, the techniques described herein relate to a cardiac implant, wherein activation of the first pump causes driving of blood out of the blood vessel.

In some aspects, the techniques described herein relate to a cardiac implant, wherein activation of the second pump causes driving of blood towards the blood vessel.

In some aspects, the techniques described herein relate to a cardiac implant, further including a stent configured to anchor the one or more lines.

In some aspects, the techniques described herein relate to a cardiac implant, wherein the stent has a tubular shape.

In some aspects, the techniques described herein relate to a cardiac implant, wherein the one or more lines are disposed at an external surface of the stent.

In some aspects, the techniques described herein relate to a cardiac implant, wherein the one or more lines are disposed at an internal surface of the stent.

In some aspects, the techniques described herein relate to a cardiac implant, wherein the blood vessel is an aorta.

In some aspects, the techniques described herein relate to a cardiac implant comprising a pump coupled to a fluid tube and configured to manage flow of fluid between a first inflatable balloon and a second inflatable balloon.

Described herein are devices, methods, and systems that facilitate the improvement of stroke volume within one or more blood vessels and/or heart chambers. restoration and/or enhancement of compliance characteristics for target blood vessels. Devices associated with the various examples of the present disclosure can include one or more fluid transfer devices (e.g., balloons and/or pumps) configured to convey a fluid between a proximal device disposed within a blood vessel and a distal device disposed outside the blood vessel. Some example systems can include a stent configured to sandwich one or more components of the implant between the stent and an inner wall of the blood vessel.

For purposes of summarizing the disclosure, certain aspects, advantages and novel features have been described. It is to be understood that not necessarily all such advantages may be achieved in accordance with any particular example. Thus, the disclosed examples may be carried out in a manner that achieves or optimizes one advantage or group of advantages as taught herein without necessarily achieving other advantages as may be taught or suggested herein.

Methods and structures disclosed herein for treating a patient also encompass analogous methods and structures performed on or placed on a simulated patient, which is useful, for example, for training; for demonstration; for procedure and/or device development; and the like. The simulated patient can be physical, virtual, or a combination of physical and virtual. A simulation can include a simulation of all or a portion of a patient, for example, an entire body, a portion of a body (e.g., thorax), a system (e.g., cardiovascular system), an organ (e.g., heart), or any combination thereof. Physical elements can be natural, including human or animal cadavers, or portions thereof; synthetic; or any combination of natural and synthetic. Virtual elements can be entirely in silica, or overlaid on one or more of the physical components. Virtual elements can be presented on any combination of screens, headsets, holographically, projected, loudspeakers, headphones, pressure transducers, temperature transducers, or using any combination of suitable technologies.

### BRIEF DESCRIPTION OF THE DRAWINGS

Various examples are depicted in the accompanying drawings for illustrative purposes and should in no way be interpreted as limiting the scope of the inventions. In addition, various features of different disclosed examples can be combined to form additional examples, which are part of this disclosure. Throughout the drawings, reference numbers may be reused to indicate correspondence between reference elements.

FIG. 1A illustrates an example representation of a heart and associated vasculature having various features relevant to one or more examples of the present inventive disclosure.

FIGS. 1B-1 and 1B-2 show detailed views of example healthy and aged/stiff aortas, respectively.

FIGS. 2A-1 and 2B-1 provide side and cross-sectional views, respectively, of a compliant blood vessel, such as an artery (e.g., aorta), experiencing expansion during the systolic phase of the cardiac cycle.

FIGS. 2A-2 and 2B-2 provide side and cross-sectional views, respectively, of a compliant blood vessel, such as an artery (e.g., aorta), experiencing contracting/recoiling during the diastolic phase of the cardiac cycle.

FIGS. 3-1 and 3-2 show a cross-sectional profile of a blood vessel that is relatively stiff, similar to the blood vessel shown in FIG. 1B-2, wherein the compliance of the vessel portion is diminished relative to the healthy aorta as shown in FIG. 1B-1.

FIG. 4 is a graph illustrating blood pressure over time in an example patient with a healthy, compliant aorta, wherein arterial blood pressure is represented as a combination of a forward systolic pressure wave and a backward diastolic pressure wave.

FIG. 5 is a graph illustrating blood pressure over time in an example patient having reduced aortic compliance.

FIGS. 6A and 6B illustrates two states of an example blood flow assist device in accordance with one or more examples.

FIG. 7 illustrates another example blood flow assist device comprising a stent 708 and/or prosthetic valve in accordance with one or more examples.

FIG. 8 provides a flowchart illustrating an example process for improving stroke volume in accordance with one or more examples.

FIG. 9 illustrates another example blood flow assist device in accordance with one or more examples.

### DETAILED DESCRIPTION

The headings provided herein are for convenience only and do not necessarily affect the scope or meaning of the claimed invention.

Although certain preferred examples are disclosed below, it should be understood that the inventive subject matter extends beyond the specifically disclosed examples to other alternative examples and/or uses and to modifications and equivalents thereof. Thus, the scope of the claims that may arise herefrom is not limited by any of the particular examples described below. For example, in any method or process disclosed herein, the acts or operations of the method or process may be performed in any suitable sequence and are not necessarily limited to any particular disclosed sequence. Various operations may be described as multiple discrete operations in turn, in a manner that may be helpful in understanding certain examples; however, the order of description should not be construed to imply that these operations are order dependent. Additionally, the structures, systems, and/or devices described herein may be embodied as integrated components or as separate components. For purposes of comparing various examples, certain aspects and advantages of these examples are described. Not necessarily all such aspects or advantages are achieved by any particular example. Thus, for example, various examples may be carried out in a manner that achieves or optimizes one advantage or group of advantages as taught herein without necessarily achieving other aspects or advantages as may also be taught or suggested herein.

Certain reference numbers are re-used across different figures of the figure set of the present disclosure as a matter of convenience for devices, components, systems, features, and/or modules having features that may be similar in one or more respects. However, with respect to any of the examples disclosed herein, re-use of common reference numbers in the drawings does not necessarily indicate that such features, devices, components, or modules are identical or similar. Rather, one having ordinary skill in the art may be informed by context with respect to the degree to which usage of common reference numbers can imply similarity between referenced subject matter. Use of a particular reference number in the context of the description of a particular figure can be understood to relate to the identified device, component, aspect, feature, module, or system in that particular figure, and not necessarily to any devices, components, aspects, features, modules, or systems identified by the same reference number in another figure. Furthermore, aspects of separate figures identified with common reference numbers can be interpreted to share characteristics or to be entirely independent of one another.

Where an alphanumeric reference identifier is used that comprises a numeric portion and an alphabetic portion (e.g., ‘10a,’ ‘10’ is the numeric portion and ‘a’ is the alphabetic portion), references in the written description to only the numeric portion (e.g., ‘10’) may refer to any feature identified in the figures using such numeric portion (e.g., ‘10a,’ ‘10b,’ ‘10c,’ etc.), even where such features are identified with reference identifiers that concatenate the numeric portion thereof with one or more alphabetic characters (e.g., ‘a,’ ‘b,’ ‘c,’ etc.). That is, a reference in the present written description to a feature ‘10’ may be understood to refer to either an identified feature ‘10a’ in a particular figure of the present disclosure or to an identifier ‘10’ or ‘10b’ in the same figure or another figure, as an example.

Certain standard anatomical terms of location are used herein to refer to the anatomy of animals, and namely humans, with respect to various examples. Although certain spatially relative terms, such as “outer,” “inner,” “upper,” “lower,” “below,” “above,” “vertical,” “horizontal,” “top,” “bottom,” and similar terms, are used herein to describe a spatial relationship of one device/element or anatomical structure to another device/element or anatomical structure, it is understood that these terms are used herein for ease of description to describe the positional relationship between element(s)/structures(s), as illustrated in the drawings. It should be understood that spatially relative terms are intended to encompass different orientations of the element(s)/structures(s), in use or operation, in addition to the orientations depicted in the drawings. For example, an element/structure described as “above” another element/structure may represent a position that is below or beside such other element/structure with respect to alternate orientations of the subject patient or element/structure, and vice-versa. It should be understood that spatially relative terms, including those listed above, may be understood relative to a respective illustrated orientation of a referenced figure.

### Vascular Anatomy and Compliance

Certain examples are disclosed herein in the context of vascular implant devices, and in particular, compliance-enhancement implant devices implanted in the aorta. However, although certain principles disclosed herein may be particularly applicable to the anatomy of the aorta, it should be understood that compliance-enhancement implant devices in accordance with the present disclosure may be implanted in, or configured for implantation in, any suitable or desirable blood vessels or other anatomy, such as the inferior vena cava.

The anatomy of the heart and vascular system is described below to assist in the understanding of certain inventive concepts disclosed herein. In humans and other vertebrate animals, the heart generally comprises a muscular organ having four pumping chambers, wherein the flow thereof is at least partially controlled by various heart valves, namely, the aortic, mitral (or bicuspid), tricuspid, and pulmonary valves. The valves may be configured to open and close in response to a pressure gradient present during various stages of the cardiac cycle (e.g., relaxation and contraction) to at least partially control the flow of blood to a respective region of the heart and/or to blood vessels (e.g., ventricles, pulmonary artery, aorta, etc.). The contraction of the various heart muscles may be prompted by signals generated by the electrical system of the heart.

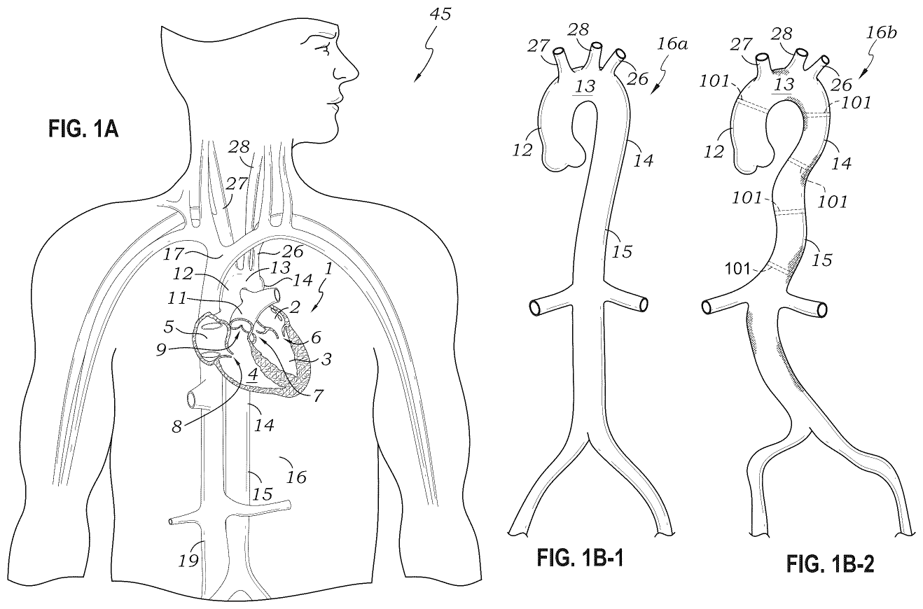

FIG. 1A illustrates an example representation of a heart 1 and associated vasculature having various features relevant to one or more examples of the present inventive disclosure. The heart 1 includes four chambers, namely the left atrium 2, the left ventricle 3, the right ventricle 4, and the right atrium 5. In terms of blood flow, blood generally flows from the right ventricle 4 into the pulmonary artery 11 via the pulmonary valve 9, which separates the right ventricle 4 from the pulmonary artery 11 and is configured to open during systole so that blood may be pumped toward the lungs and close during diastole to prevent blood from leaking back into the heart from the pulmonary artery 11. The pulmonary artery 11 carries deoxygenated blood from the right side of the heart to the lungs. The pulmonary artery 11 includes a pulmonary trunk and left and right pulmonary arteries that branch off of the pulmonary trunk, as shown.

The tricuspid valve 8 separates the right atrium 5 from the right ventricle 4. The tricuspid valve 8 generally has three cusps/leaflets and may generally close during ventricular contraction (i.e., systole) and open during ventricular expansion (i.e., diastole). The mitral valve 6 generally has two cusps/leaflets and separates the left atrium 2 from the left ventricle 3. The mitral valve 6 is configured to open during diastole so that blood in the left atrium 2 can flow into the left ventricle 3, and, when functioning properly, closes during systole to prevent blood from leaking back into the left atrium 2. The aortic valve 7 separates the left ventricle 3 from the ascending aortic trunk 12. The aortic valve 7 is configured to open during systole to allow blood leaving the left ventricle 3 to enter the aorta 12, and close during diastole to prevent blood from leaking back into the left ventricle 3.

The heart valves may generally comprise a relatively dense fibrous ring, referred to herein as the annulus, as well as a plurality of leaflets or cusps attached to the annulus. Generally, the size of the leaflets or cusps may be such that when the heart contracts the resulting increased blood pressure produced within the corresponding heart chamber forces the leaflets at least partially open to allow flow from the heart chamber. As the pressure in the heart chamber subsides, the pressure in the subsequent chamber or blood vessel may become dominant and press back against the leaflets. As a result, the leaflets/cusps come in apposition to each other, thereby closing the flow passage. Disfunction of a heart valve and/or associated leaflets (e.g., pulmonary valve disfunction) can result in valve leakage and/or other health complications.

The vasculature of the human body, which may be referred to as the circulatory system, cardiovascular system, or vascular system, contains a complex network of blood vessels with various structures and functions and includes various veins (venous system) and arteries (arterial system). Generally, arteries, such as the aorta 16, carry blood away from the heart, whereas veins, such as the inferior and superior venae cavae, carry blood back to the heart.

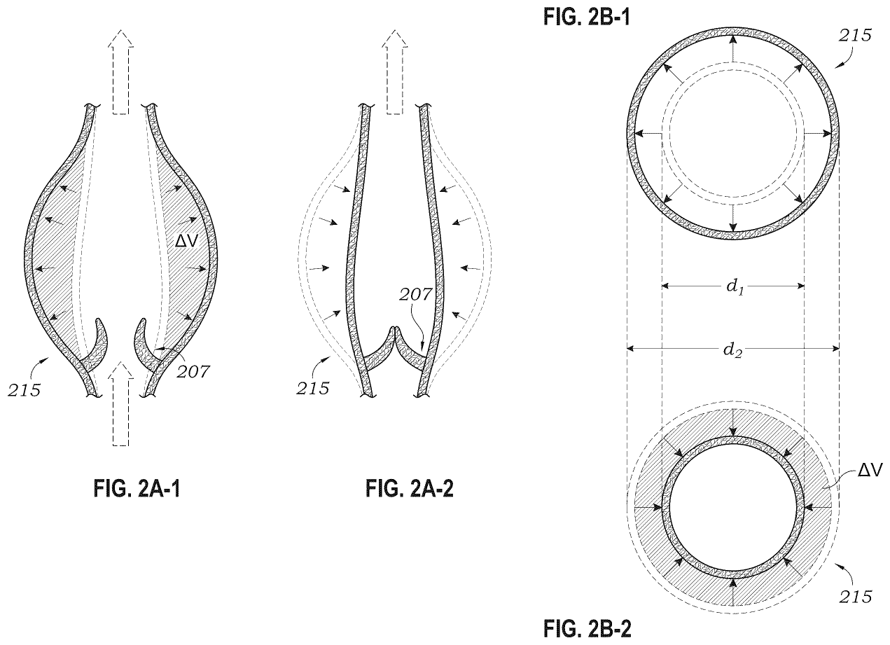

FIGS. 1B-1 and 1B-2 show detailed views of example healthy 16a and aged/stiff 16b aortas, respectively. The aorta 16 is a compliant arterial blood vessel that buffers and conducts pulsatile left ventricular output and contributes the largest component of total compliance of the arterial tree. The aorta 16 includes the ascending aorta 12, which begins at the opening of the aortic valve 7 in the left ventricle of the heart and is sometimes referred to as the aortic ‘trunk.’ The ascending aorta 12 and pulmonary trunk 11 twist around each other, causing the aorta 12 to start out posterior to the pulmonary trunk 11, but end by twisting to its right and anterior side. Among the various segments of the aorta 16, the ascending aorta 12 is relatively frequently affected by aneurysms and dissections, often requiring open heart surgery to be repaired. The transition from the ascending aorta 12 to the aortic arch 13 is at the pericardial reflection on the aorta 16. At the root of the ascending aorta 12, the lumen has three small pockets between the cusps of the aortic valve and the wall of the aorta, which are called the aortic sinuses or the sinuses of Valsalva. The left aortic sinus contains the origin of the left coronary artery and the right aortic sinus likewise gives rise to the right coronary artery. Together, these two arteries supply the heart.

As mentioned above, the aorta is coupled to the heart 1 via the aortic valve 7, which leads into the ascending aorta 12 and gives rise to the innominate artery 27, the left common carotid artery 28, and the left subclavian artery 26 along the aortic arch 13 before continuing as the descending thoracic aorta 14 and further the abdominal aorta 15. References herein to the aorta may be understood to refer to the ascending aorta 12 (also referred to as the “ascending thoracic aorta”), aortic arch 13, descending or thoracic aorta 14 (also referred to as the “descending thoracic aorta”), abdominal aorta 15, or other arterial blood vessel or portion thereof.

Arteries, such as the aorta 16, may utilize blood vessel compliance (e.g., arterial compliance) to store and release energy through the stretching of blood vessel walls. The term “compliance” is used herein according to its broad and ordinary meaning, and may refer to the ability of an arterial blood vessel or prosthetic implant device to distend, expand, stretch, or otherwise deform in a manner as to increase in volume in response to increasing transmural pressure, and/or the tendency of a blood vessel (e.g., artery) or prosthetic implant device, or portion thereof, to recoil toward its original dimensions (e.g., lesser volume) as transmural pressure decreases.

Arterial compliance facilitates perfusion of organs in the body with oxygenated blood from the heart. Generally, a healthy aorta and other major arteries in the body are at least partially elastic and compliant, such that they can act as a reservoir for blood, filling up with blood when the heart contracts during systole and continuing to generate pressure and push blood to the organs of the body during diastole. In older individuals and patients suffering from heart failure and/or atherosclerosis, compliance of the aorta and other arteries can be diminished to some degree or lost. Such reduction in compliance can reduce the supply of blood to the organs of the body due to the decrease in blood flow during diastole. Among the risks associated with insufficient arterial compliance, a significant risk presented in such patients is a reduction in blood supply to the heart muscle itself. For example, during systole, generally little or no blood may flow in the coronary arteries and into the heart muscle due to the contraction of the heart which holds the heart at relatively high pressures. During diastole, the heart muscle generally relaxes and allows flow into the coronary arteries. Therefore, perfusion of the heart muscle relies on diastolic flow, and therefore on aortic/arterial compliance.

A healthy aorta 16a, as shown in FIG. 1B-1, runs along a generally straight path, whereas an aged and/or stiffened aorta 16b, as shown in FIG. 1B-2, can run along a more tortuous, curved path. That is, the aorta tends to change in shape as a function of age, resulting in higher degrees of curvature or tortuosity, as developed gradually over time. Such change in shape of the blood vessel can be associated with the vasculature of the subject becoming less elastic. As such conditions develop, arterial blood pressure (e.g., left-ventricular afterload) can become more pulsatile, which can have deleterious effects, such as the thickening of the left ventricle (LV) muscle, and insufficient perfusion of the heart. Insufficient perfusion of the heart muscle can lead to and/or be associated with heart failure. Heart failure is a clinical syndrome characterized by certain symptoms, including breathlessness, ankle swelling, fatigue, and others. Heart failure may be accompanied by certain signs, including elevated jugular venous pressure, pulmonary crackles, and peripheral edema, for example, which may be caused by structural and/or functional cardiac abnormality. Such conditions can result in reduced cardiac output and/or elevated intra-cardiac pressures at rest or during stress.

Examples of the present disclosure provide compliance-enhancing implant devices, which may be implanted in one or more locations in a compromised aorta and/or other vessel(s). For example, FIG. 1B-2 shows example positions of compliance-enhancing implant devices 101 implanted in various areas of an aorta 16b. Embodiments of the present disclosure provide elastic reshaping of a target blood vessel, such as the aorta, in a manner as to produce a volume differential between high-and low-pressure states, thereby mimicking conditions of a stretchy, healthy blood vessel.

FIGS. 2A-1 and 2B-1 provide side and cross-sectional views, respectively, of a compliant blood vessel 215, such as an artery (e.g., aorta), experiencing expansion during the systolic phase of the cardiac cycle. FIGS. 2A-2 and 2B-2 provide side and cross-sectional views, respectively, of the compliant blood vessel 215 radially contracting/recoiling during the diastolic phase of the cardiac cycle. As understood by those having ordinary skill in the art, the systolic phase of the cardiac cycle is associated with the pumping phase of the left ventricle, while the diastolic phase of the cardiac cycle is associated with the filling phase of the left ventricle. As identified in FIGS. 2A-1 and 2B-2, with proper arterial compliance, a change in volume ΔV will generally occur in an artery between high- and low-pressure phases of the cardiac cycle. With respect to the aorta, as shown in FIGS. 2A-1 and 2B-1, as blood is pumped into the aorta 215 through the aortic valve 207, the pressure in the aorta increases and the diameter of at least a portion of the aorta expands. While the blood vessel 215 is shown forming a bulge, dilation of the blood vessel 215 may involve a generally uniform expansion of the blood vessel 215 rather than a localized bulge. A first portion of the blood entering the aorta 215 during systole may pass through the aorta during the systolic phase, while a second portion (e.g., approximately half of the total blood volume) may be stored in the expanded volume ΔV (see FIG. 2A-1) caused by compliant stretching of the blood vessel, thereby storing energy for contributing to perfusion during the diastolic phase. A compliant aorta may generally stretch with each heartbeat, such that the diameter of at least a portion of the aorta expands.

The tendency of the arteries to stretch in response to pressure as a result of arterial compliance may have a significant effect on perfusion and/or blood pressure in some patients. For example, arteries with relatively higher compliance may be conditioned to more easily deform than lower-compliance arteries under the same pressure conditions. Compliance (C) may be calculated using the following equation, where ΔV is the change in volume (e.g., in mL) of the blood vessel, and ΔP is the pulse pressure from systole to diastole (e.g., in mmHg):

$$C = \frac{Δ V}{Δ P} \qquad ( 1 )$$

Aortic stiffness and reduced compliance can lead to elevated systolic blood pressure, which can in turn lead to elevated intracardiac pressures, increased afterload, and/or other complications that can exacerbate heart failure. Aortic stiffness further can lead to reduced diastolic flow, which can lead to reduced coronary perfusion, decreased cardiac supply, and/or other complications that can likewise exacerbate heart failure.

Arterial compliance restoration devices, methods, and concepts disclosed herein may be generally described in the context of the ascending aorta. However, it should be understood that such devices, methods and/or concepts may be applicable in connection with any other artery or blood vessel.

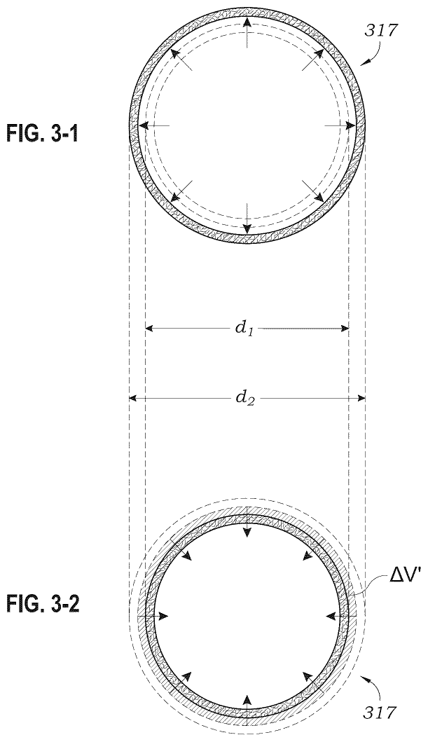

FIGS. 3-1 and 3-2 show a cross-sectional profile of a blood vessel 317 that is relatively stiff, similar to the blood vessel 16b shown in FIG. 1B-2, wherein the compliance of the vessel portion 317 is diminished relative to the healthy aorta 16a as shown in FIG. 1B-1. Due to the stiffness of the blood vessel wall, the blood vessel 317 may expand a relatively limited amount ΔV′ between diastole (shown in FIG. 3-2) and systole (shown in FIG. 3-1). That is, during systole, the increased fluid pressure within the blood vessel 317 may result only in a relatively small and/or negligible expansion of the diameter of the blood vessel 317, as shown with respect to the difference between the contracted diameter d₁ and the expanded diameter d₂ in FIGS. 3-1 and 3-2. Due to the limited expansion of the blood vessel 317, the change in volume ΔV' in the blood vessel between phases of the cardiac cycle may likewise be limited, and therefore relatively little energy is stored in the blood vessel wall in high-pressure conditions and returned to the blood circulation during low-pressure conditions, resulting in more pulsatile blood flow compared to healthy, compliant aortic tissue.

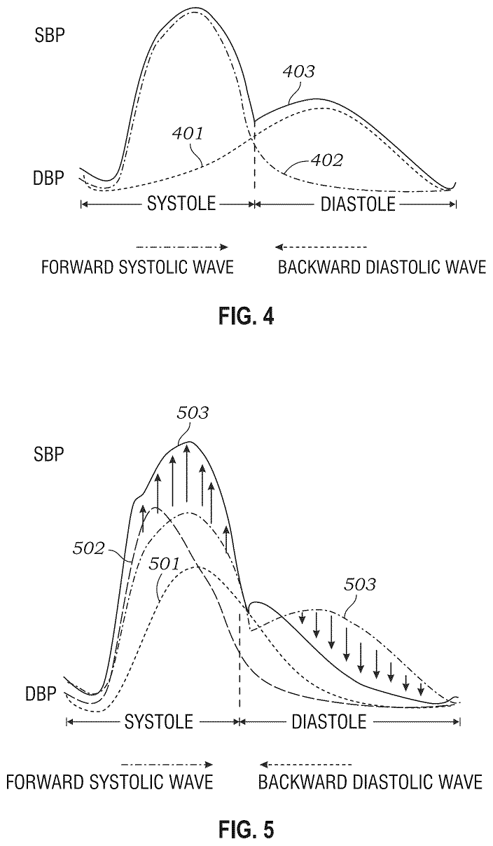

FIG. 4 is a graph illustrating blood pressure over time in an example patient with a healthy, compliant aorta, wherein arterial blood pressure is represented as a combination of a forward systolic pressure wave 402 and a backward diastolic pressure wave 401. The combination of the systolic wave 402 and the diastolic wave 401 are approximately represented by the waveform 403.

FIG. 5 is a graph illustrating blood pressure over time in an example patient having reduced aortic compliance. The graph of FIG. 5 shows, for reference purposes, the example combined wave 403 shown in FIG. 4. When low compliance is exhibited, less energy may be stored in the aorta compared to a healthy patient. Therefore, the systolic waveform 502 may demonstrate increased pressure during the systolic phase relative to a patient having normal compliance, while the diastolic waveform 501 may demonstrate reduced pressure during the diastolic phase relative to a patient having normal compliance. Therefore, the resulting combined waveform 503 may represent an increase in the systolic peak and a drop in the diastolic pressure, which may cause various health complications. For example, the change in waveform may impact the workload on the left ventricle and may adversely affect coronary profusion.

In view of the health complications that may be associated with reduced arterial compliance, as described above, it may be desirable in certain patients and/or under certain conditions, to at least partially alter compliance properties of the aorta or other artery or blood vessel, or otherwise alter/control flow therein, in order to improve cardiac/organ health. Disclosed herein are various devices and methods for at least partially restoring and/or increasing compliance in a blood vessel, such as the aorta. Certain examples disclosed herein achieve restoration of arterial compliance through the use of implantable and/or expandable implants configured to be implanted at least partially within a blood vessel and at least partially outside the blood vessel. For example, such implants may be configured to expand in accordance with elastic features/characteristics thereof and store energy during higher-pressure periods of the cardiac cycle (e.g., during the systolic phase). During lower-pressure periods (e.g., during the diastolic phase), such implant devices can contract/deflate to reshape the target blood vessel in a manner as to reduce a volume thereof to thereby return the stored energy to the circulation and increase flow through the vessel.

As the vasculature of a subject and/or of a subject blood vessel becomes less elastic, arterial blood pressure (e.g. left-ventricular afterload) can become more pulsatile. This can have deleterious effects such as thickening of the left ventricle (LV) muscle and/or diastolic heart failure.

In some examples, devices of the present disclosure include inflatable and/or expandable implants configured to be implant at least partially within a target blood vessel (e.g., the aorta) and/or at least partially outside the target blood vessel. A liquid-filled volume (e.g., implant and/or balloon) may be implanted partially within the aorta, and partly outside the aorta, such that, for each cycle (e.g., heartbeat), a distal and/or extraaortic portion of the liquid-filled volume can expand as increased aortic blood presses compresses a proximal and/or intraaortic portion. The distal and/or extraaortic portion of the volume can reinflate the proximal and/or intraaortic portion with the liquid as aortic blood pressure decreases.

Devices of the present disclosure may be delivered and/or implanted at least partially within a target blood vessel, such as in the aorta (e.g., aortic trunk, descending thoracic, or abdominal aorta), using percutaneous, transcatheter, and/or other minimally-invasive means, such as through a direct minimally-invasive path to the exterior of the aorta through the back and/or flank of the patient. With respect to transcatheter and/or percutaneous procedures, implants of the present disclosure may be advanced to the target area of the blood vessel through the vasculature.

Devices of the present disclosure may be delivered via the vasculature. In some examples, a branching blood vessel may be cut by deploying occluding devices prior to transecting the branching blood vessel completely (e.g., by an ablation) from within the vasculature rather than creating a minimally invasive access from, for example, the patient's back and/or flank.

### Stroke Volume Improvement Implants

Examples described herein relate to devices for improving stroke volume in patients. Patients with congestive heart failure (CHF) can suffer from impaired stroke volume (SV). Ventricle assist devices may be utilized to improve cardiac output. In some cases, assist devices delivery may involve surgical implantation procedures for attachment to the heart (e.g., attachment to the external heart wall to apply external squeezing forces). In some cases, CHF patients can also require aortic valve replacements.

Some ventricle assist devices described herein can advantageously be configured for non-surgical procedures and/or transcatheter delivery. In some examples, a ventricle assist device may be configured comprise a replacement valve (e.g., an aortic valve replacement) and/or can be configured to improve stroke volume. In some examples, only a single procedure may be required to deliver an assist device and/or a valve replacement device.

Some examples herein relate to devices for improving stroke volume and/or coronary flow (e.g., in CHF patients). An example device (e.g., stroke volume improvement device) can comprise two or more synchronized volume-changing components, which can include balloons and/or pumps. While some examples herein are described with respect to the aorta, aortic valve, left atrium, and/or left ventricle, example devices may be configured for delivery and/or use at other areas and/or valves of the heart.

In some examples, a device can comprise a first balloon and/or other volume device (e.g., in the aorta) fluidly connected to a second balloon and/or volume device (e.g., in the left ventricle) via a flexible tube. During diastole, the first balloon may be configured to inflate and/or the second balloon may be configured to deflate. During systole, the first balloon may be configured to deflate and/or the second balloon may be configured to inflate.

An inflation fluid or gas may be transitioned between the first balloon and the second balloon via the flexible tube. For example, the same fluid and/or gas may fill the first balloon and the second balloon alternately.

In some examples, a device can comprise a first pump (e.g., in the aorta) and/or a second pump (e.g., in the left ventricle). During diastole, the first pump may be open and/or in an open state and/or the second pump may be closed and/or in a closed state. During systole, the first pump may be closed and/or the second pump may be open. The first pump and/or the second pump may be controlled electrically via one or more electric wires. In some examples, one or more conductive wires may extend between the first pump and the second pump to facilitate transmission of electrical signals between the first pump and the second pump.

A first volume device (e.g., balloon and/or pump) and/or a second volume device (e.g., a balloon and/or pump) may be connected to a prosthetic heart valve (e.g., a prosthetic aortic valve) and/or may be disposed on either side of the prosthetic heart valve. In some examples, a device may comprise one or more pressure sensors. For example, the device may comprise a pressure sensor at a volume device disposed in the left ventricle and/or aorta. The first volume device and/or second volume device may be configured to transition (e.g., between a deflated and inflated state and/or between a closed and/or open state) in response to pressure data from the pressure sensor.

The present disclosure can relate to systems, devices, and methods for adding back and/or increasing compliance in the aorta or other arterial (or venous) blood vessel(s) to provide improved perfusion of the heart muscle and/or other organ(s) of the body. Examples of the present disclosure can include inflatable and/or expandable implants which may be referred to herein as “accumulators” and/or “hydraulic accumulators.” The term “accumulator” is used herein in accordance with its plain and ordinary meaning and may refer to any balloon, container, bag, vessel, stent, and/or similar device configured to hold one or more gases and/or fluids. In some examples, an accumulator may be configured allow one or more contained gases and/or fluids to move between a proximal end and/or a distal end of the accumulator.

The accumulator can function as a hydraulic accumulator and/or may be configured to smooth out peaks (e.g., lower maximum blood pressure) and/or troughs (e.g., increase minimum blood pressure) in aortic blood pressure. A proximal and/or intraaortic part of the accumulator may be disposed between a wall of the aorta and a lined stent (e.g. similar to stents used for aortic aneurysms and/or parallel stent grafting). Increased blood pressure through the blood vessel can cause deflation and/or activation of the proximal part, which can cause inflation of a distal part and/or can cause driving of blood and/or fluid out of the blood vessel and/or into an adjoining chamber. The stent may also advantageously provide anchoring for the implant within the blood vessel and/or heart valve (e.g., aortic valve).

Examples described herein relate to devices for smoothing out peaks and troughs in aortic blood pressure. Examples devices can be liquid-filled and/or can be configured for placement partially within the aorta and/or other blood vessel and/or partially outside the aorta and/or other blood vessel. In some examples, an implant may be configured to adjust and/or change on a periodic basis. For example, during each heartbeat, a distal portion of the implant (e.g., outside the aorta) may expand as increased blood pressure compresses a proximal portion (e.g., inside the aorta) and/or presses the liquid to the distal portion. Additionally or alternatively, the distal portion may be configured to reinflate the proximal portion with the liquid as blood pressure decreases.

As the implants of the present disclosure produce complaint blood vessel volume change by manipulating/reshaping the native blood vessel walls, compliance can be increased in the target blood vessel without requiring blood vessel grafting or resection. Therefore, compared to blood flow solutions involving blood vessel grafting/resection, examples of the present disclosure can provide a solution that avoids the risks that may be associated with cutting of the vessel and/or devices grafted in/to such vessels, which may present risk of rupture and blood leakage. Hazards associated with extravascular arterial blood leakage, such as within the abdominal and/or chest cavity, can include the risk of serious injury or death.

As described above, desirable diastolic flow in arterial blood vessels is enabled by the decrease in cross-sectional area/volume of the blood vessels when transitioning from higher-pressure conditions (e.g., systole) to lower-pressure conditions (e.g., diastole). Where the relevant blood vessel has become stiff and non-compliant, stretching/expanding and subsequent contraction/shrinking of the blood vessel to cause the desired change in area/volume of the blood vessel may be limited due to the perimeter/wall of the blood vessel resisting stretching. Examples of the present disclosure provide implants that cause a change in cross-sectional area/volume of a target blood vessel without requiring stretching in the blood vessel wall. Rather, such cyclical change in blood vessel area/volume can be achieved through manipulation of the shape (e.g., cross-sectional shape) of the target blood vessel, wherein a transition between blood vessel shapes occurring in response to changing pressure conditions can reduce and increase the area/volume of the blood vessel in a cyclical manner to promote more even flow of blood through the blood vessel throughout the cardiac cycle.

With respect to a blood vessel having a relatively fixed perimeter, wherein the blood vessel wall does not expand sufficiently due to stiffness and/or other factors of non-compliance, generally, the greatest area/volume of the blood vessel may be present/achieved when the blood vessel wall forms a circular cross-sectional shape, which may maximize the cross-sectional area of the blood vessel.

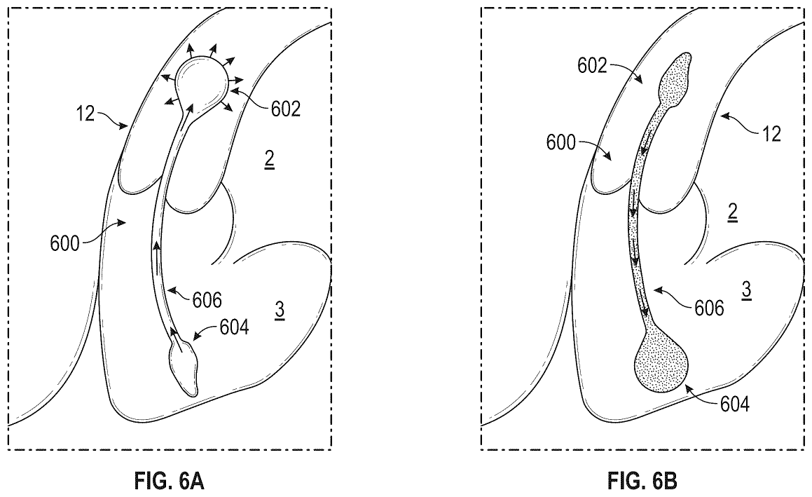

FIGS. 6A and 6B illustrates two states of an example blood flow assist device 600 and/or cardiac implant in accordance with one or more examples. FIG. 6A illustrates the device 600 during diastole and/or in response to diastolic pressure and FIG. 6B illustrates the device 600 during systole and/or in response to systolic pressure. The device 600 may be configured to transfer fluid between various components of the device 600.

The device 600 can comprise two or more synchronized and/or alternating volume devices and/or fluid transfer devices (e.g., balloons and/or pumps) configured to transition between multiple states alternately. For example, the device 600 can comprise one or more fluid and/or gas pumps configured to transition between open and closed states. In some examples, the device 600 may comprise a first volume device 602 and/or fluid transfer device placed in a first anatomical area (e.g., the aorta 12 and/or aortic arch) and/or a second volume device 604 (e.g., balloon) and/or fluid transfer device placed in a second anatomical area (e.g., the left ventricle 3).

As shown in FIG. 6A, during diastole and/or a first anatomical event, the second volume device 604 can deflate, close (e.g., a pump closing to drive fluid), and/or drive fluid and/or gas from the second volume device 604 to and/or via a fluid tube 606 (e.g., fluid line) and/or to the first volume device 602. Movement of fluid and/or gas from, for example, the left ventricle 3 to the aorta 12 can allow for improved left ventricle 3 filling from the left atrium 2 while the first volume device 602 inflates, opens (e.g., a pump opening to receive fluid), and/or receives fluid and/or gas from the fluid tube 606 and/or from the second volume device 604. Deflation of the second volume device 604 may be configured to create an under-pressure in the aorta 12 (e.g., above the aortic valve and/or at or near an aortic arch) and/or can assist in blood flow by pulling blood towards the aorta 12 with a suction effect. In some examples, deflation of the first volume device 602 may be configured to cause inflation of the second volume device 604 and/or deflation of the second volume device 604 may be configured to cause inflation of the first volume device 602.

As shown in FIG. 6B, during systole and/or a second anatomical event, the second volume device 604 can 602 inflate, open (e.g., a pump opening to receive fluid), and/or receive fluid and/or gas from the fluid tube 606 and/or first volume device 602 and/or the first volume device 602 can deflate, close (e.g., a pump closing to drive fluid), and/or drive fluid and/or gas from the first volume device 602 to the fluid tube 606 and/or to the second volume device 604 to improve stroke volume. Deflation of the first volume device 602 can cause suction of blood towards, for example, the aorta. Increase in volume of the second volume device 604 can be caused by negative pressure (e.g., a vacuum effect) resulting from the transition from diastole to systole.

In some examples, the first volume device 602 and/or the second volume device 604 may be fluidly coupled to each other via the fluid tube 606 and/or fluid line. The fluid tube 606 may have a generally flexible and/or fluid-tight structure. In some examples, the fluid tube 606 (e.g., shaft and/or line) can enable the same volume of inflation fluid (e.g., saline, gas, etc.) to flow between the first volume device 602 and/or the second volume device 604. In some examples, fluid may be conveyed via the fluid tube 606 and/or some amount of fluid may be retained at least partially within the fluid tube 606. For example, excess fluid following inflation of the first volume device 602 and/or second volume device 604 may be retained within the fluid tube 606. In some examples, the first volume device 602 and/or second volume device 604 may not be simultaneously inflated and/or may not be simultaneously fully inflated. The first volume device 602 and the second volume device 604 may be simultaneously partially deflated and/or simultaneously fully deflated as fluid is transferred between the volume devices and/or is disposed within the fluid tube 606.

In some examples, the first volume device 602 and/or the second volume device 604 may comprise fluid pumps configured to convey fluid between each other. For example, the first volume device 602 and/or the second volume device 604 may be synchronized via electrical wires extending between the first volume device 602 and/or the second volume device 604 (e.g., across and/or through the aortic valve). The wires may be coupled to a battery and/or within the body and/or may be configured to extend outside the body to an external power source. The first volume device 602 and/or second volume device 604 may be configured to be powered and/or operated via electrical power, signals, and/or current. For example, current and/or signals transmitted via the electrical wires may be configured to allow for controlled movement of the first volume device 602 and/or second volume device 604. In some examples, a surgeon may be able to selectively cause opening and/or closing of the first volume device 602 and/or second volume device 604 as needed via controls electrically connected to the electrical wires. In some examples, the first volume device 602 and/or second volume device 604 may be configured to be operated on a cycle and/or periodic basis automatically via the electrical wires and/or other power and/or communication source. The first volume device 602 and/or second volume device 604 may be configured to be powered and/or operated via wireless transmitters and/or power sources. For example, a wireless battery disposed outside the patient's body may be configured to supply power to the first volume device 602 and/or second volume device 604 disposed within the patient's body.

In some examples, the first volume device 602 and/or second volume device 604 may be configured to open (e.g., to a generally circular form) to allow fluid to enter the first volume device 602 and/or second volume device 604 and/or may be configured to close (e.g., to the deflated form shown in FIGS. 6A and 6B) to cause driving of fluid from the first volume device 602 and/or second volume device 604 and/or to other components of the device 600. The first volume device 602 and/or second volume device 604 may be configured to drive fluid through anatomical pathways. For example, the first volume device 602 may be disposed within an aorta 12 and/or other blood vessel and/or may be configured to drive fluid out of the aorta 12 and/or other blood vessel (e.g., through the aortic valve). The second volume device 604 may be disposed within a left ventricle 3 and/or outside the aorta 12 and/or other blood vessel and/or may be configured to drive fluid towards and/or into the aorta 12 and/or other blood vessel (e.g., through the aortic valve).

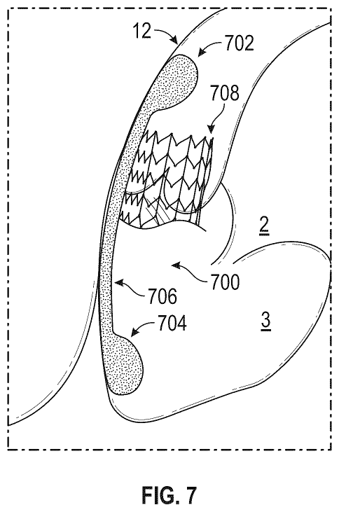

FIG. 7 illustrates another example blood flow assist device 700 comprising a stent 708 and/or prosthetic valve in accordance with one or more examples. In some cases, patients experiencing CHF can require a prosthetic valve replacement (e.g., a prosthetic aortic valve replacement). In some examples, a stent 708 and/or valve replacement device may be incorporated into the blood flow assist device 700 as shown in FIG. 7.

In some examples, the device 700 can comprise a first volume device 702 (e.g., an inflatable balloon and/or fluid-driving pump) configured for placement at least partially within a first anatomical area, which can include an aorta 12 and/or an aortic arch. The device 700 can additionally comprise a second volume device 704 (e.g., an inflatable balloon and/or fluid-driving pump) configured for placement at least partially within a second anatomical area, which can include a left ventricle 3. The first volume device 702 and/or the second volume device 704 may be interconnected by a fluid tube 706 extending between the first volume device 702 and/or the second volume device 704. The fluid tube 706 may have a generally thin and/or elongate form and/or may be configured to convey fluid and/or gas bi-directionally between the first volume device 702 and/or the second volume device 704. In some examples, the fluid tube 706 may have a generally thin and/or at least partially expandable form.

The stent 708 may be configured to attach and/or anchor to an outer surface of the fluid tube 706. In some examples, the fluid tube 706 may be configured to attach and/or anchor to an outer surface of the stent 708, as shown in FIG. 7. However, the fluid tube 706 may be configured to extend through an inner lumen of the stent 708 and/or may be configured to attach and/or anchor to an inner surface of the stent 708. In some examples, the stent 708 may be configured to anchor the fluid tube 706 and/or various lines (e.g., fluid lines and/or electrical lines) extending between the first volume device 702 and the second volume device 704. For example, the various lines may be configured to attach to an outer and/or inner surface of the stent 708 such that the lines may be secured away from a central portion of the aorta 12 and/or aortic valve. In some examples, the stent 708 may have a generally tubular shape configured to approximate a tubular shape of the aorta 12 and/or other blood vessel. The stent 708 may form an inner lumen to allow blood flow through the stent 708. In some examples, the stent 708 may comprise stent walls in a cylindrical and/or tubular form configured to press against the walls of the aorta 12 and/or blood vessel.

The stent 708 may advantageously be configured to prevent migration of the first volume device 702, second volume device 704, and/or fluid tube 706. For example, the stent 708 may be configured to anchor at least partially within the aorta 12, the aortic valve, and/or other anatomy. In some examples, the stent 708 may be at least partially expandable and/or may be configured to expand from a compressed form (e.g., during delivery) to an expanded form in which the sides of the stent 708 are expanded into contact with tissue around the stent 708. In some examples, the stent 708 may be configured for placement at least partially within a heart valve (e.g., the aortic valve).

In some examples, the stent 708 may comprise a prosthetic valve. For example, the stent 708 may comprise one or more leaflets extending at least partially across an inner lumen of the stent 708. In some examples, the stent 708 may comprise a docking station and/or may be configured to dock one or more prosthetic valves at an inner lumen and/or inner surface of the stent 708.

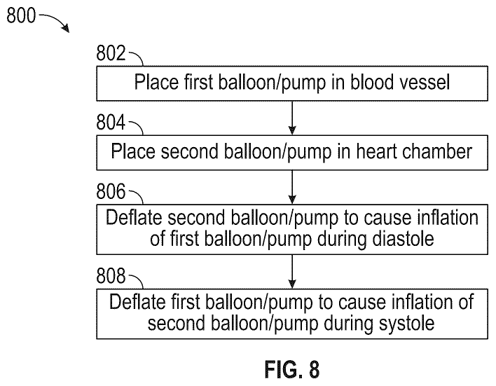

FIG. 8 provides a flowchart illustrating an example process 800 for improving stroke volume in accordance with one or more examples. Steps of the process 800 may be performed in any order.

At step 802, the process 800 involves placing a first balloon and/or pump of an implant in a blood vessel (e.g., the aorta). The implant can be delivered to the implant in any suitable manner, which can include a percutaneous and/or trans-femoral approach (e.g., via a catheter). The implant may comprise the first balloon and/or pump, a second balloon and/or pump, a fluid tube, and/or an anchoring stent.

In some examples, some components of the implant can be operated by control circuitry and/or a power source embedded therein. For example, one or more pumps of the implant may be operated electrically. In some examples, the implant can be powered wirelessly from an external power transmitter (e.g., disposed outside the body). For example, the implant can be powered via electromagnetic and/or radio frequency (RF) energy.

In some examples, the implant can comprise one or more pressure sensors at the first balloon and/or pump and/or at the second balloon and/or pump. The one or more pressure sensors can be configured to control the transition between states of the implant according to pressure difference signals detected during systole and/or diastole. In some examples, the device can be controlled by electrocardiogram (ECG) electrodes, which can be configured to synchronize a transition between states according to ECG signals indicative of the systolic and/or diastolic phases.

At step 804, the process 800 involves placing the second balloon and/or pump in a chamber of the heart (e.g., the left ventricle). The second balloon and/or pump may be connected and/or tethered to the first balloon and/or pump via one or more wires and/or tubes. In some examples, the one or more wires and/or tubes may be configured to extend through a valve (e.g., aortic valve) of the heart.

At step 806, the process 800 involves deflating and/or deactivating the second balloon and/or pump to cause inflation and/or activation of the first balloon and/or pump. In some examples, deflation and/or deactivation of the second balloon and/or pump may be performed during diastole and/or other cardiac event.

At step 808, the process 800 involves deflating and/or deactivating the first balloon and/or pump to cause inflation and/or activation of the second balloon and/or pump. In some examples, deflation and/or deactivation of the first balloon and/or pump may be performed during systole and/or other cardiac event.

The above method(s) can be performed on a living animal or on a simulation, such as on a cadaver, cadaver heart, anthropomorphic ghost, simulator (e.g., with body parts, heart, tissue, etc. being simulated).

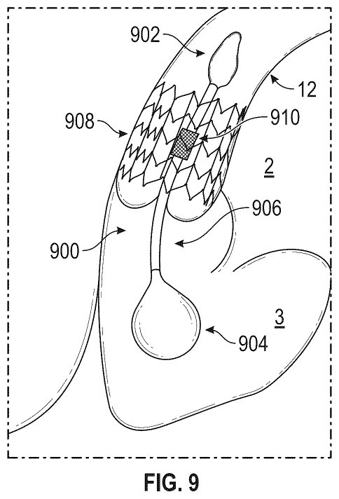

FIG. 9 illustrates another example blood flow assist device 900 in accordance with one or more examples. The device 900 can comprise a stent 908 and/or prosthetic valve. In some cases, patients experiencing CHF can require a prosthetic valve replacement (e.g., a prosthetic aortic valve replacement). In some examples, the stent 908 and/or valve replacement device may be incorporated into the blood flow assist device 900 as shown in FIG. 9.

In some examples, the device 900 can comprise a first volume device 902 (e.g., an inflatable balloon) configured for placement at least partially within a first anatomical area, which can include an aorta 12 and/or an aortic arch. The device 900 can additionally comprise a second volume device 904 (e.g., an inflatable balloon) configured for placement at least partially within a second anatomical area, which can include a left ventricle 3. The first volume device 902 and/or the second volume device 904 may be interconnected by a fluid tube 906 (e.g., tether) extending between the first volume device 902 and/or the second volume device 904. The fluid tube 906 may have a generally thin and/or elongate form and/or may be configured to convey fluid and/or gas bi-directionally between the first volume device 902 and/or the second volume device 904. In some examples, the fluid tube 906 may have a generally thin and/or at least partially flexible and/or expandable form.

The device 900 can be configured to move between different states and/or forms. For example, the first volume device 902 and/or second volume device 904 may be configured to inflate/deflate in an alternating manner. For example, deflation of the first volume device 902 may cause and/or may be caused by inflation of the second volume device 904 and/or vice versa. FIG. 9 illustrates the device 900 in a first state in which the first volume device 902 is deflated and the second volume device 904 is inflated, however the first volume device 902 and/or the second volume device 904 may be configured to change forms.

The fluid tube 906, first volume device 902, and/or second volume device 904 may be configured to extend at least partially through an inner lumen of the stent 908. In some examples, the fluid tube 906 may be disposed approximately at a center position within the inner lumen of the stent 908. For example, the tube 906 and/or stent 908 may comprise one or more arms configured to hold the tube 906 away from a frame and/or from walls of the stent 908 and/or generally along a central axis of the stent 908.

In some examples, the device 900 may comprise a fluid pump 910 coupled to the tube 906 and/or configured to create and/or manage flow through the tube 906 and/or between the first volume device 902 and/or second volume device 904. The pump 910 may be disposed at least partially within the tube 906 and/or may be coupled at and/or around an exterior of the tube 906. The pump 910 may comprise any suitable device and/or mechanism configured to control and/or manage flow within the tube 906 and/or towards the first volume device 902 and/or second volume device 904. In some examples, the pump 910 may be configured to generate flow in multiple directions. For example, the pump 910 may be configured to push and/or pump fluid and/or gas towards the first volume device 902 and/or towards the second volume device 904. In some examples, the pump 910 may be configured to promote flow towards the first volume device 902 and/or the second volume device 904 in an alternating manner. For example, the pump 910 may be configured to direct flow within the tube 906 towards the first volume device 902 during diastole to cause inflation of the first volume device 902, deflation of the second volume device 904, and/or increased blood into the coronary arteries. Similarly, the pump 910 may be configured to direct flow within the tube 906 towards the second volume device 904 during systole to cause inflation of the second volume device 904, deflation of the first volume device 902, and/or reduction of left ventricular volume and/or increased blood flow from the left ventricle 3 to the aorta 12. The pump 910 may comprise any suitable mechanism(s), which can include various hydraulic devices, for example an Archimedes' screw. The pump 910 can comprise one or more rotational elements configured to direct flow in a first direction (e.g., towards the first volume device 902) when rotating a first direction and/or to direct flow in a second direction (e.g., towards the second volume device 904) when rotating in a second direction.

In some examples, the pump 910 may be coupled to and/or may be in electronic communication with one or more sensors. The one or more sensors may be configured to sense blood pressure changes and/or changes in cardiac cycles. For example, the one or more sensors may be configured to detect and/or identify diastole and/or systole. The one or more sensors may be configured to communicate to the pump 910 the cardiac cycle and/or the pump 910 may be configured to modify fluid flow within the tube 906 based on signals from the one or more sensors. For example, in response to a sensor signal identifying diastole, the pump 910 may be configured to direct fluid flow towards the first volume device 902. In response to a sensor signal identifying systole, the pump 910 may be configured to direct fluid flow towards the second volume device 904. The one or more sensors may be coupled to the pump 910 and/or tube 906 and/or may be external to the pump 910 and/or tube 906. The one or more sensors may be positioned such that the one or more sensors may be exposed to blood flow within the first anatomical area and/or second anatomical area.

The pump 910 may comprise a power source (e.g., one or more batteries) and/or may be coupled to a power source. In some examples, the power source may be disposed outside the body and/or may be coupled to the pump 910 via one or more wires. However, the power source may be disposed within the body and/or attached to and/or near the pump 910.

The fluid tube 906 may be configured to extend through an inner lumen of the stent 908 and/or may be configured to attach and/or anchor to an inner surface of the stent 908. In some examples, the stent 908 may be configured to anchor the fluid tube 906 and/or various lines (e.g., fluid lines and/or electrical lines) extending between the first volume device 902 and the second volume device 904. For example, the various lines may be configured to attach to an outer and/or inner surface of the stent 908 such that the lines may be secured away from a central portion of the aorta 12 and/or aortic valve. In some examples, the stent 908 may have a generally tubular shape configured to approximate a tubular shape of the aorta 12 and/or other blood vessel. The stent 908 may form an inner lumen to allow blood flow through the stent 908. In some examples, the stent 908 may comprise stent walls in a cylindrical and/or tubular form configured to press against the walls of the aorta 12 and/or blood vessel.

The stent 908 may advantageously be configured to prevent migration of the first volume device 902, second volume device 904, and/or fluid tube 906. For example, the stent 908 may be configured to anchor at least partially within the aorta 12, the aortic valve, and/or other anatomy. In some examples, the stent 908 may be at least partially expandable and/or may be configured to expand from a compressed form (e.g., during delivery) to an expanded form in which the sides of the stent 908 are expanded into contact with tissue around the stent 908. In some examples, the stent 908 may be configured for placement at least partially within a heart valve (e.g., the aortic valve 12).

In some examples, the stent 908 may comprise a prosthetic valve. For example, the stent 908 may comprise one or more leaflets extending at least partially across an inner lumen of the stent 908. In some examples, the stent 908 may comprise a docking station and/or may be configured to dock one or more prosthetic valves at an inner lumen and/or inner surface of the stent 908.

### Additional Description of Examples

Provided below is a list of examples, each of which may include aspects of any of the other examples disclosed herein. Furthermore, aspects of any example described above may be implemented in any of the numbered examples provided below.

Depending on the example, certain acts, events, or functions of any of the processes or algorithms described herein can be performed in a different sequence, may be added, merged, or left out altogether. Thus, in certain examples, not all described acts or events are necessary for the practice of the processes.

Example 1: A cardiac implant comprising a first fluid transfer device configured for placement at least partially within a blood vessel of a heart, a second fluid transfer device configured for placement outside the blood vessel, and one or more lines interconnecting the first fluid transfer device and the second fluid transfer device.

Example 2: The cardiac implant of any example herein, in particular example 1, wherein the first fluid transfer device comprises a first inflatable balloon.

Example 3: The cardiac implant of any example herein, in particular example 2, wherein the one or more lines comprise a fluid tube.

Example 4: The cardiac implant of any example herein, in particular example 3, wherein the second fluid transfer device comprises a second inflatable balloon.

Example 5: The cardiac implant of any example herein, in particular example 4, wherein the fluid tube is configured to convey a fluid between the first inflatable balloon and the second inflatable balloon.

Example 6: The cardiac implant of any example herein, in particular example 5, wherein deflation of the first inflatable balloon causes inflation of the second inflatable balloon.

Example 7: The cardiac implant of any example herein, in particular example 5, wherein deflation of the second inflatable balloon causes inflation of the first inflatable balloon.

Example 8: The cardiac implant of any example herein, in particular example 1, wherein the first fluid transfer device comprises a first pump.

Example 9: The cardiac implant of any example herein, in particular example 8, wherein the one or more lines comprise one or more electrical wires.

Example 10: The cardiac implant of any example herein, in particular example 9, wherein the second fluid transfer device comprises a second pump.

Example 11: The cardiac implant of any example herein, in particular example 10, wherein the one or more electrical wires are configured to convey electrical signals between the first pump and the second pump.

Example 12: The cardiac implant of any example herein, in particular example 10, wherein activation of the first pump causes driving of blood out of the blood vessel.

Example 13: The cardiac implant of any example herein, in particular example 10, wherein activation of the second pump causes driving of blood towards the blood vessel.

Example 14: The cardiac implant of any example herein, in particular example 1, further comprising a stent configured to anchor the one or more lines.

Example 15: The cardiac implant of any example herein, in particular example 14, wherein the stent has a tubular shape.

Example 16: The cardiac implant of any example herein, in particular example 15, wherein the one or more lines are disposed at an exterior surface of the stent.

Example 17: The cardiac implant of any example herein, in particular example 15, wherein the one or more lines are disposed at an interior surface of the stent.

Example 18: The cardiac implant of any example herein, in particular example 5, comprising a pump coupled to the fluid tube and configured to manage flow of the fluid between the first inflatable balloon and the second inflatable balloon.

Example 19: A method comprising delivering a first fluid transfer device configured at least partially within a blood vessel of a heart, delivering a second fluid transfer device outside the blood vessel, and deflating or deactivating the second fluid transfer device to cause inflation of the first fluid transfer device.

Example 20: The method of any example herein, in particular example 19, wherein the first fluid transfer device and the second fluid transfer device are delivered percutaneously.

Example 21: The method of any example herein, in particular example 19, further comprising deflating or deactivating the first fluid transfer device to cause inflation of the second fluid transfer device.

Example 22: The method of any example herein, in particular example 19, wherein the first fluid transfer device comprises a first inflatable balloon.

Example 23: The method of any example herein, in particular example 22, wherein the first fluid transfer device and the second fluid transfer device are connected via one or more lines.

Example 24: The method of any example herein, in particular example 23, wherein the one or more lines comprise a fluid tube.

Example 25: The method of any example herein, in particular example 24, wherein the second fluid transfer device comprises a second inflatable balloon.

Example 26: The method of any example herein, in particular example 25, wherein the fluid tube is configured to convey a fluid between the first inflatable balloon and the second inflatable balloon.

Example 27: The method of any example herein, in particular example 19, wherein the first fluid transfer device comprises a first pump.

Example 28: The method of any example herein, in particular example 27, wherein the first fluid transfer device and the second fluid transfer device are connected via one or more lines.

Example 29: The method of any example herein, in particular example 28, wherein the one or more lines comprise one or more electrical wires.

Example 30: The method of any example herein, in particular example 29, wherein the second fluid transfer device comprises a second pump.

Example 31: The method of any example herein, in particular example 30, wherein the one or more electrical wires are configured to convey electrical signals between the first pump and the second pump.

Example 32: The method of any example herein, in particular example 30, wherein activation of the first pump causes driving of blood out of the blood vessel.

Example 33: The method of any example herein, in particular example 30, wherein activation of the second pump causes driving of blood towards the blood vessel.

Example 34: The method of any example herein, in particular example 19, wherein the blood vessel is an aorta.

The treatment techniques, methods, steps, etc. described or suggested herein or in references incorporated herein can be performed on a living animal or on a non-living simulation, such as on a cadaver, cadaver heart, anthropomorphic ghost, simulator (e.g., with the body parts, tissue, etc. being simulated), etc.

Any of the various systems, devices, apparatuses, etc. in this disclosure can be sterilized (e.g., with heat, radiation, ethylene oxide, hydrogen peroxide, etc.) to ensure they are safe for use with patients, and the methods herein can comprise sterilization of the associated system, device, apparatus, etc. (e.g., with heat, radiation, ethylene oxide, hydrogen peroxide, etc.).

Conditional language used herein, such as, among others, “can,” “could,” “might,” “may,” “e.g.,” and the like, unless specifically stated otherwise, or otherwise understood within the context as used, is intended in its ordinary sense and is generally intended to convey that certain examples include, while other examples do not include, certain features, elements and/or steps. Thus, such conditional language is not generally intended to imply that features, elements and/or steps are in any way required for one or more examples or that one or more examples necessarily include logic for deciding, with or without author input or prompting, whether these features, elements and/or steps are included or are to be performed in any particular example. The terms “comprising,” “including,” “having,” and the like are synonymous, are used in their ordinary sense, and are used inclusively, in an open-ended fashion, and do not exclude additional elements, features, acts, operations, and so forth. Also, the term “or” is used in its inclusive sense (and not in its exclusive sense) so that when used, for example, to connect a list of elements, the term “or” means one, some, or all of the elements in the list. Conjunctive language such as the phrase “at least one of X, Y and Z,” unless specifically stated otherwise, is understood with the context as used in general to convey that an item, term, element, etc. may be either X, Y or Z. Thus, such conjunctive language is not generally intended to imply that certain examples require at least one of X, at least one of Y and at least one of Z to each be present.

It should be appreciated that in the above description of examples, various features are sometimes grouped together in a single example, Figure, or description thereof for the purpose of streamlining the disclosure and aiding in the understanding of one or more of the various inventive aspects. This method of disclosure, however, is not to be interpreted as reflecting an intention that any claim require more features than are expressly recited in that claim. Moreover, any components, features, or steps illustrated and/or described in a particular example herein can be applied to or used with any other example(s). Further, no component, feature, step, or group of components, features, or steps are necessary or indispensable for each example. Thus, it is intended that the scope of the inventions herein disclosed and claimed below should not be limited by the particular examples described above, but should be determined only by a fair reading of the claims that follow.

It should be understood that certain ordinal terms (e.g., “first” or “second”) may be provided for ease of reference and do not necessarily imply physical characteristics or ordering. Therefore, as used herein, an ordinal term (e.g., “first,” “second,” “third,” etc.) used to modify an element, such as a structure, a component, an operation, etc., does not necessarily indicate priority or order of the element with respect to any other element, but rather may generally distinguish the element from another element having a similar or identical name (but for use of the ordinal term). In addition, as used herein, indefinite articles (“a” and “an”) may indicate “one or more” rather than “one.” Further, an operation performed “based on” a condition or event may also be performed based on one or more other conditions or events not explicitly recited.

Unless otherwise defined, all terms (including technical and scientific terms) used herein have the same meaning as commonly understood by one of ordinary skill in the art to which example examples belong. It be further understood that terms, such as those defined in commonly used dictionaries, should be interpreted as having a meaning that is consistent with their meaning in the context of the relevant art and not be interpreted in an idealized or overly formal sense unless expressly so defined herein.

The spatially relative terms “outer,” “inner,” “upper,” “lower,” “below,” “above,” “vertical,” “horizontal,” and similar terms, may be used herein for ease of description to describe the relations between one element or component and another element or component as illustrated in the drawings. It be understood that the spatially relative terms are intended to encompass different orientations of the device in use or operation, in addition to the orientation depicted in the drawings. For example, in the case where a device shown in the drawing is turned over, the device positioned “below” or “beneath” another device may be placed “above” another device. Accordingly, the illustrative term “below” may include both the lower and upper positions. The device may also be oriented in the other direction, and thus the spatially relative terms may be interpreted differently depending on the orientations.

Unless otherwise expressly stated, comparative and/or quantitative terms, such as “less,” “more,” “greater,” and the like, are intended to encompass the concepts of equality. For example, “less” can mean not only “less” in the strictest mathematical sense, but also, “less than or equal to.”

What is claimed is:

1. A cardiac implant comprising:
a first balloon sized for placement at least partially within a blood vessel of a heart;
a second balloon sized for placement outside the blood vessel;
a tube interconnecting the first balloon and the second balloon to convey fluid between the first balloon and the second balloon; and
a stent coupled to the tube to anchor the tube within the blood vessel.

2. The cardiac implant of claim 1, wherein deflation of the first balloon causes inflation of the second balloon.

3. The cardiac implant of claim 2, wherein deflation of the second balloon causes inflation of the first balloon.

4. The cardiac implant of claim 1, further comprising an electronic pump coupled to the tube to manage flow of fluid between the first balloon and the second balloon.

5. The cardiac implant of claim 1, further comprising a first electronic pump coupled to the first balloon to cause the first balloon to expand or contract.

6. The cardiac implant of claim 5, further comprising one or more electrical lines coupled to the first electronic pump.

7. The cardiac implant of claim 5, further comprising a second electronic pump coupled to the second balloon.

8. The cardiac implant of claim 1, wherein the tube is coupled to an outside surface of the stent to position the tube between the stent and a wall of the blood vessel.

9. The cardiac implant of claim 1, wherein the tube is sized to extend through a valve of the heart.

10. The cardiac implant of claim 1, wherein the stent is sized for placement at least partially within a valve of the heart.

11. The cardiac implant of claim 1, wherein the stent comprises one or more arms sized to position the tube at a central position of the stent.

12. A cardiac implant comprising:
a first inflation device sized for placement at least partially within a blood vessel of a heart;
a first electronic pump coupled to the first inflation device to cause the first inflation device to expand or contract;
a second inflation device sized for placement outside the blood vessel; and
a tube interconnecting the first inflation device and the second inflation device to convey fluid between the first inflation device and the second inflation device.

13. The cardiac implant of claim 12, further comprising a stent coupled to the tube to anchor the tube within the blood vessel.

14. The cardiac implant of claim 12, further comprising a second electronic pump coupled to the tube to manage flow of fluid between the first inflation device and the second inflation device.

15. The cardiac implant of claim 12, further comprising one or more electrical lines coupled to the first electronic pump.

16. The cardiac implant of claim 12, further comprising a second electronic pump coupled to the second inflation device.

17. A cardiac implant for assisting blood flow comprising:
a first inflation device sized for placement at least partially within a blood vessel of a heart near a valve of the heart;
a second inflation device sized for placement outside the blood vessel and within an anatomical chamber adjacent to the valve of the heart;
a tube interconnecting the first inflation device and the second inflation device to convey fluid between the first inflation device and the second inflation device, the tube sized to extend through the valve of the heart; and
a stent coupled to the tube to anchor the tube within the blood vessel.

18. The cardiac implant of claim 17, wherein the tube is coupled to an outside surface of the stent to position the tube between the stent and a wall of the blood vessel.

19. The cardiac implant of claim 17, wherein the stent is sized for placement at least partially within a valve of the heart.

20. The cardiac implant of claim 17, wherein the stent comprises one or more arms sized to position the tube at a central position of the stent.
